(19) United States
(12) Patent Application Publication
(10) Pub. No.: US 2003/0013944 A1
(43) Pub. Date: Jan. 16, 2003

(54) Prevention of Bell's palsy

(76) Inventor: Anuthep Benja-Athon, New York, NY (US)

Correspondence Address: ANUTHEP BENJA-ATHON, M.D., Pain Management, Rehabilitation Medicine, Acupuncture Physical Therapy, 210 East 36th Street, New York, NY, 10016, US

(21) Appl. No.: 09/905,497

(22) Filed: Jul. 16, 2001

(51) Int. Cl. A61B 5/00

(52) U.S. Cl. 600/300; 128/923

(57) ABSTRACT

A system and method for foreseeing the occurrence of Bell's palsy comprises statistical techniques of analyzing and establishing the relationships based on the definition, characteristics and properties of Bell's palsy and humans susceptible to Bell's palsy, conditions, findings, symptoms, signs, events and circumstances associate with, occurring and leading to Bell's palsy. The system comprises humans to detect the definition, characteristics and properties of Bell's palsy and medical history and profile of humans susceptible to Bell's palsy and the conditions, findings, symptoms, signs, events and circumstances associate with, occurring prior and leading to the onset of Bell's palsy. Statistical techniques are used to analyze and predict the above relationships and correlations. Statistical techniques are use to analyze and predict the results and outcomes of treatments.

### FIELD OF INVENTION

A method and system for foretelling the coming of Bell's palsy.

### BACKGROUND OF THE INVENTION

The first objective is to foresee or predict the occurrence of Bell's palsy.

The second objective is to detect individual events, occurrences, developments and progressions associate with, prior and leading to Bell's palsy.

The third objective is to prevent Bell's palsy.

Bell's palsy is the paralysis or weakness of the unilateral or bilateral facial muscles caused by injury to the facial nerve of unknown cause(s).

Every 13 minutes one American was afflicted by Bell's palsy. (Adour, K. K. 1972) This potentially facial-disfiguring disease of epic proportion for a person has an incidence rate of about 23 per 100,000 people per year. (Hauser W. A. 1971). This figure translates to a staggering number of about 44,000 Americans per year.

Like stroke, Bell's palsy struck people in their prime of life and all ages were not spared. A person might get up one morning and usually find that one side of the face was paralyzed. A catastrophe resulting in utmost physical challenges to the victim. “A willing mind is imprisoned in an unwilling body™”, the paralyzed face not only caused life-long disfigurement and psychological burden and scar but also many disorders of and problems with sensations, taste, mastication and eating, vision and hearing. On the paralyzed face, the patient could neither smile nor whistle, had abnormal tearing, eyelids which failed to close causing injuries and damage to the eye, deranged taste, food trapped in the cheek, ringing in the ear and hearing disturbances, etc. Overnight, a career and livelihood were left in ruin or destroyed.

Since its first description by Sir Charles Bell in 1821, Bell's palsy permanently branded about 55%-90% afflicted victims with above life-long facial deformity and disorders. In large percentage of the 44,000 people per annum, patients would have a prodrome of about one week before the onset of Bell's palsy. The prodromal warning comprises various findings, events, circumstances, symptoms and signs heralding the coming of Bell's palsy. This warning were such as, but not limited to, an episode of preceding infection, pain in the head, neck and face, pain in or about the ear, earache, excessive tearing at onset, dry eye, taste change or abnormal taste, tingling or numbness in cheek, mouth or tongue, and hyperacusis. This warning was not recognized by anyone including the victims, patients and physicians. There are no system for detecting, predicting, monitoring, analyzing, terminating the events and the sequences prior and leading to the onset and occurrence of Bell's palsy. The victims and patients passively laid in the path of this medical catastrophe which could have been either avoided or prevented.

In this modern society wherein there are systems of surveillance on hurricanes and earthquakes, there is yet a system for foreseeing the herald of this tragic illness so that avoidance and prevention of Bell's palsy can be taken. Given the knowledge described herein, no victim would passively wait to be struck by Bell's palsy.

Once Bell's palsy struck, it was too late for a patient since damage would have already occurred to the facial nerve (see 2001 Bell's Palsy, Cause, Prevention and Treatment™, A. Benja-Athon, M.D. @ www.MuscleJointNerve.com). The outcomes could mean a life-long disfigurement of the face and aforementioned disorders and problems.

In summary, people including physicians are totally handicapped and lack the ability to deal with the events prior to the onset of Bell's palsy.

This present invention provides said avoidance and prevention. It provides the solution to above 181-year problems and dilemma.

### SUMMARY OF THE INVENTION

Since its description in 1821, the prevention Bell's palsy are not known. People are afflicted with this disease and are totally helpless.

A method and means for foreseeing the occurrence of Bell's palsy comprises statistical techniques of analyzing and establishing the relationships based on the definition, characteristics and properties of Bell's palsy and humans susceptible to Bell's palsy, and conditions, findings, symptoms, signs, events and circumstances associate with, occurring prior and leading to Bell's palsy henceforth known as prodrome. Said means comprises means for detecting said definition, characteristics and properties of Bell's palsy and humans susceptible to Bell's palsy and prodrome. Said means for detecting are such as, but not limited to, people sustaining, involving and knowledgeable on said definition, characteristics and properties of Bell's palsy and humans susceptible to Bell's palsy, and prodrome. Means for foreseeing comprises statistical means for analyzing, correlating, predicting and nullfying. Said statistical means are such as, but not limited to, statistical inference means for analyzing, predicting and producing the above relationship. Said statistical means also correlates said definition, characteristics and properties of Bell's palsy and humans susceptible to Bell's palsy, and prodrome. Statistical techniques are used to exclude a relationship between conditions, findings, symptoms, signs, events and circumstances neither leading to nor associate with Bell's palsy and said definition, characteristics, properties of Bell's palsy and said human, definition, characteristics and properties of Bell's palsy and humans susceptible to Bell's palsy and prodrome comprises statistical inference means for excluding said relationship. Statistical techniques are used to analyze the results and outcomes of the treatments of said conditions, findings, symptoms, signs, events and circumstances associate with, occurring prior and leading to the onset of Bell's palsy.

### BRIEF DESCRIPTION OF THE DRAWING

The schematic description of the present invention.

### DESCRIPTION OF THE PREFERRED EMBODIMENTS

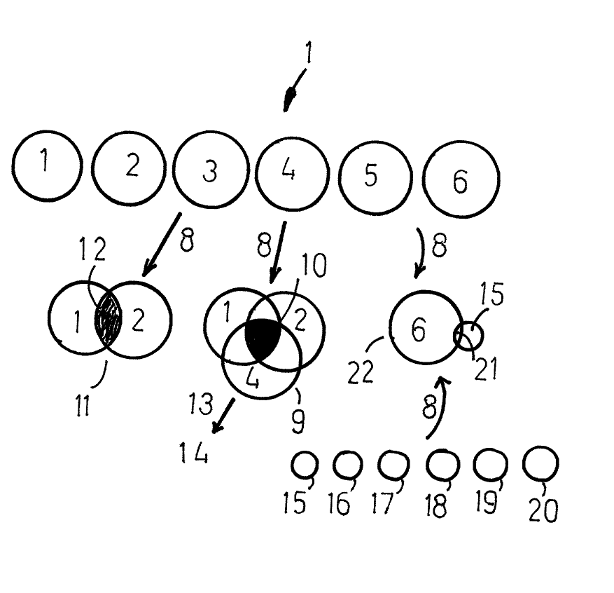

FIG. 1 depicts the means for foreseeing and method 1 of the present invention.

A means for foreseeing the occurrence of idiopathic facial paralysis or palsy otherwise known as Bell's palsy comprises definition of Bell's palsy, characteristics and properties of Bell's palsy and means for identifying the characteristics and properties of a human susceptible to Bell's palsy.

The characteristics and properties of humans susceptible to Bell's palsy are the demography, medical history, profile of humans susceptible to Bell's palsy such as, but not limited to, various diseases including diabetes and hypertension, males and females of certain ages, and pregnancy.

The conditions, findings, symptoms, signs, events and circumstances leading to and associate with Bell's palsy are represented by circles, number 1-6, respectively. Persons involving, sustaining and witnessing conditions, findings, symptoms, signs, events and circumstances 1-6 are such as, but not limited to, potential victims, patients, friends and families, physicians and other health care providers who are experiencing or knowledgeable on said conditions 1, findings 2, symptoms 3, signs 4, events 5, circumstances 6 and any combination thereof.

Conditions 1 are such as, but not limited to, infection, seasons; findings 2 are such as, but not limited to, preceding viral infections or dormant viral infections; symptoms 3 are such as, but not limited to, pain in the head, neck and face, pain in or about the ear, earache, excessive tearing at onset, dry eye, taste change or abnormal taste, tingling or numbness in cheek, mouth or tongue, and hyperacusis; signs 4 are such as, but not limited to, abnormal tearing, redness of the eardrum, inflamed throat, sinuses or upper respiratory airway; events 5 are such as, but not limited to, surgery or comprised immune system; circumstances 6 are such as, but not limited to, exposure to cold air or draft and stress.

A individual statistical weights and significances may be assigned to conditions 1, findings 2, symptoms 3, signs 4, events 5, circumstances 6 and any combination thereof.

Statistical techniques 8 are such as, but not limited to, statistical inference including correlation analysis, retrogression analysis, chi square and student's t test that predicts, establishes and specifies said relationships among said definition, characteristics, properties of Bell's palsy and said human, conditions, findings, symptoms, signs, events and circumstances 1-6.To obtain good statistical correlation 12 involves the analysis of condition(s) 1 and finding(s) 2 in 11 setting. However, better correlation 10 is caused by statistical correlation 9 of condition(s) 1, finding(s) 2 and sign(s) 4. Various permutations 13 of said definitions, characteristics, properties of Bell's palsy and said human, conditions 1, findings 2, symptoms 3, signs 4, events 5 and circumstances 6 and statistical techniques 8 can be applied to improve said means for foreseeing the occurrence of Bell's palsy 14 as per routine statistical analysis.

Similarly, said definition, characteristics, properties of Bell's palsy and said human, conditions 1, findings 2, symptoms 3, signs 4, events 5 and circumstances 6 are correlated using statistical techniques 8 to derive said results.

The parallel conditions 15, findings 16, symptoms 17, signs 18, events 19 and circumstances 20 are neither associate with, occur before nor lead to Bell's palsy. These are those which are different from and beyond said conditions 1, findings 2, symptoms 3, signs 4, events 5, circumstances 6.

Statistical techniques 8 are applied to exclude or rule out a relationship between conditions 15, findings 16, symptoms 17, signs 18, events 19, circumstances 20 and any combination thereof and said definition, characteristics, properties of Bell's palsy and said human, and conditions 1, findings 2, symptoms 3, signs 4, events 5, circumstances 6 and any combination thereof.

To obtain or yield poor statistical correlation or null relationship 21 from statistical analysis 22 between conditions 15, findings 16, symptoms 17, signs 18, events 19, circumstances 20 and any combination thereof and said definition, characteristics, properties of Bell's palsy and said human, and conditions 1, findings 2, symptoms 3, signs 4, events 5, circumstances 6 and any combination thereof involves statistical techniques 8 and steps as described above.

The above means and method lead to a recommendation for a course of action or non-action wherein treatments are either given or withheld in said converse to said people to treat conditions 1, findings 2, symptoms 3, signs 4, events 5 and circumstances 6 in order to avoid Bell's palsy. The treatments are withheld in conditions 15, findings 16, symptoms 17, signs 18, events 19, circumstances 20.

To analyze the effectiveness of treatments, results and outcomes of said treatments of conditions 1, findings 2, symptoms 3, signs 4, events 5 and circumstances 6 prior to Bell's palsy, the results and outcome of said individual treatments, modalities or combination of said treatments or modalities can be analyzed using said statistical techniques as described above.

Although various preferred embodiments of this invention have been described, it will be appreciated by those skilled in the art that adaptations and variations may be made without departing from the spirit of the invention and the scope of the claims.

Although Bell's palsy was used as an example of the preferred embodiments in this invention, it will be appreciated by those skilled in the medicine and science that adaptation, application and use can be made for and extrapolated to other diseases, illnesses and disorders affecting human and other living species without departing from the spirit of the invention and the scope of the claims.

1. A means for foreseeing the occurrence of idiopathic facial paralysis or palsy otherwise known as Bell's palsy in an individual adaptable to detect and correlate individual events, occurrences, developments and progressions henceforth known as phenomena associate with, occurring prior and leading to the onset of said disease and its associated symptoms, signs and consequences, to monitor the progression and regression of said phenomena, to prevent Bell's palsy, to lead to a recommendation for a course of action or non-action wherein the analysis of said phenomena predicts, foresees, monitors, prevents and deals with said phenomena and the converse comprises:
definition of Bell's palsy;
characteristics and properties of Bell's palsy;
means for identifying the characteristics and properties of a human susceptible to Bell's palsy;
conditions, findings, symptoms, signs, events and circumstances leading to Bell's palsy;
conditions, findings, symptoms, signs, events and circumstances associate with Bell's palsy;
means for detecting said definition, characteristics and properties of Bell's palsy and said human, said conditions, findings, symptoms, signs, events and circumstances associate with, occurring prior and leading to the onset of Bell's palsy;
means for analyzing the relationships between said definition, characteristics, properties of Bell's palsy and said human, said conditions, findings, symptoms, signs, events and circumstances;
means for correlating said definition, characteristics, properties of Bell's palsy and said human, conditions, findings, symptoms, signs, events and circumstances;
means for predicting a null relationship between symptoms, signs, events and circumstances neither leading to nor associate with Bell's palsy and said definition, characteristics, properties of Bell's palsy and said human, said conditions, findings, symptoms, signs, events and circumstances associate with, occurring prior and leading to the onset of Bell's palsy;
means for excluding a relationship between said symptoms, signs, events and circumstances neither leading to nor associate with Bell's palsy and said definition, characteristics, properties of Bell's palsy and said human, said conditions, findings, symptoms, signs, events and circumstances associate with, occurring prior and leading to the onset of Bell's palsy;
means for analyzing the effectiveness of treatments including modalities on the results and outcomes of treating said conditions, findings, symptoms, signs, events and circumstances associate with, prior and leading to the onset of Bell's palsy.

2. The means for foreseeing the occurrence of idiopathic facial paralysis according to claim 1 wherein said means for identifying the characteristics and properties of a human susceptible to Bell's palsy is the demography of said human.

3. The means for foreseeing the occurrence of idiopathic facial paralysis according to claim 1 wherein said means for identifying the characteristics and properties of a human susceptible to Bell's palsy is the medical history and profile of said human.

4. The means for foreseeing the occurrence of idiopathic facial paralysis according to claim 1 wherein said means for identifying the characteristics and properties of a human susceptible to Bell's palsy are humans sustaining, involving and knowledgeable on the medical history, demography and profile of a human susceptible to Bell's palsy.

5. The means for foreseeing the occurrence of idiopathic facial paralysis according to claim 1 wherein said means for identifying the characteristics and properties of a human susceptible to Bell's palsy are humans sustaining, involving and knowledgeable on the conditions, findings, symptoms, signs, events and circumstances associate with, prior and leading to the onset of Bell's palsy and Bell's palsy.

6. The means for foreseeing the occurrence of idiopathic facial paralysis according to claim 1 wherein said means for detecting said definition, characteristics and properties of Bell's palsy and said human, said conditions, findings, symptoms, signs, events and circumstances associate with, occurring prior and leading to the onset of Bell's palsy comprises statistical inference means for correlating a plurality of said definition, characteristics, properties of Bell's palsy and said human, conditions, findings, symptoms, signs, events and circumstances associate with, prior and leading to Bell's palsy.

7. The means for foreseeing the occurrence of idiopathic facial paralysis according to claim 1 wherein said means for detecting said definition, characteristics and properties of Bell's palsy and said human, said conditions, findings, symptoms, signs, events and circumstances associate with, occurring prior and leading to the onset of Bell's palsy comprises persons involving with said conditions, findings, symptoms, signs, events and circumstances.

8. The means for foreseeing the occurrence of idiopathic facial paralysis according to claim 1 wherein said means for detecting said definition, characteristics and properties of Bell's palsy and said human, said conditions, findings, symptoms, signs, events and circumstances associate with, occurring prior and leading to the onset of Bell's palsy comprises persons sustaining or witnessing said conditions, findings, symptoms, signs, events and circumstances.

9. The means for foreseeing the occurrence of idiopathic facial paralysis according to claim 1 wherein said means for analyzing the relationships between said definition, characteristics, properties of Bell's palsy and said human, said conditions, findings, symptoms, signs, events and circumstances comprises statistical inference means for predicting, establishing and specifying said relationships.

10. The means for foreseeing the occurrence of idiopathic facial paralysis according to claim 1 wherein said means for analyzing the relationships between said definition, characteristics, properties of Bell's palsy and said human, said conditions, findings, symptoms, signs, events and circumstances comprises retrogression analysis means for predicting, establishing and specifying said relationships.

11. The means for foreseeing the occurrence of idiopathic facial paralysis according to claim 1 wherein said means for correlating said definition, characteristics, properties of Bell's palsy and said human, conditions, findings, symptoms, signs, events and circumstances comprises correlation analysis means of correlating said definition, characteristics, properties of Bell's palsy and said human, said conditions, findings, symptoms, signs, events and circumstances associate with, prior and leading to the onset of Bell's palsy.

12. The means for foreseeing the occurrence of idiopathic facial paralysis according to claim 1 wherein said means for predicting a null relationship between symptoms, signs, events and circumstances neither leading to nor associate with Bell's palsy and said definition, characteristics, properties of Bell's palsy and said human, said conditions, findings, symptoms, signs, events and circumstances associate with, occurring prior and leading to the onset of Bell's palsy comprises statistical inference means for nullifying said relationship.

13. The means for foreseeing the occurrence of idiopathic facial paralysis according to claim 1 wherein said means for excluding a relationship between said symptoms, signs, events and circumstances neither leading to nor associate with Bell's palsy and said definition, characteristics, properties of Bell's palsy and said human, said conditions, findings, symptoms, signs, events and circumstances associate with, occurring prior and leading to the onset of Bell's palsy comprises statistical inference means for excluding said relationship.

14. The means for foreseeing the occurrence of idiopathic facial paralysis according to claim 1 wherein said means for analyzing the effectiveness of treatments including modalities on the results and outcomes of treating said conditions, findings, symptoms, signs, events and circumstances associate with, prior and leading to the onset of Bell's palsy comprises the results and outcomes arising from patients treated with individual treatment and modality and without said treatment and modality.

15. The means for foreseeing the occurrence of idiopathic facial paralysis according to claim 1 wherein said means for analyzing the effectiveness of treatments including modalities on the results and outcomes of treating said conditions, findings, symptoms, signs, events and circumstances associate with, prior and leading to the onset of Bell's palsy comprises the results and outcomes arising from patients treated with combination of said treatments and modalities and without said treatments and modalities.

16. A method for foreseeing the occurrence of idiopathic facial paralysis or palsy otherwise known as Bell's palsy in an individual adaptable to detect and correlate individual events, occurrences, developments and progressions henceforth known as phenomena associate with, occurring prior and leading to the onset of disease and its associated symptoms, signs and consequences, to monitor the progression and regression of said phenomena, to prevent Bell's palsy, to lead to a recommendation for a course of action or non-action wherein analysis of said phenomena predicts, foresees, monitors, prevents and deals with said phenomena and the converse associate with, prior and leading to Bell's palsy comprises:
process of defining Bell's palsy;
process of including Bell's palsy;
process of identifying the characteristics and properties of Bell's palsy;
process of identifying the characteristics and properties of a human susceptible to Bell's palsy;
process of identifying conditions, findings, symptoms, signs, events and circumstances leading to Bell's palsy;
process of identifying conditions, findings, symptoms, signs, events and circumstances associate with Bell's palsy;
process of including conditions, findings, symptoms, signs, events and circumstances leading to Bell's palsy;
process of including conditions, findings, symptoms, signs, events and circumstances associate with Bell's palsy;
process of analyzing the relationships between said characteristics and properties of Bell's palsy and said human, said conditions, findings, symptoms, signs, events and circumstances;
process of correlating said characteristics and properties of Bell's palsy and said human, said conditions, findings, symptoms, signs, events and circumstances;
process of detecting said characteristics and properties of Bell's palsy and said human, said conditions, findings, symptoms, signs, events and circumstances associate with, occurring prior and leading to the onset of Bell's palsy;
process of predicting a null relationship between symptoms, signs, events and circumstances neither leading to nor associate with Bell's palsy and said definition, characteristics, properties of Bell's palsy and said human, said conditions, findings, symptoms, signs, events and circumstances associate with, occurring prior and leading to the onset of Bell's palsy;
process of excluding a relationship between said symptoms, signs, events and circumstances neither leading to nor associate with Bell's palsy and said definition, characteristics, properties of Bell's palsy and said human, said conditions, findings, symptoms, signs, events and circumstances associate with, occurring prior and leading to the onset of Bell's palsy; and
process of analyzing the effectiveness of treatments including modalities on the results and outcomes of treating said conditions, findings, symptoms, signs, events and circumstances associate with, occurring prior and leading to the onset of Bell's palsy.

17. The method for foreseeing the occurrence of idiopathic facial paralysis according to claim 16 wherein process of analyzing the effectiveness of treatments including modalities on the results and outcomes of treating said conditions, findings, symptoms, signs, events and circumstances associate with, prior and leading to the onset of Bell's palsy comprises the process of analyzing the results and outcomes arising from patients treated with individual treatment or modality and without said treatment or modality.

18. The method for foreseeing the occurrence of idiopathic facial paralysis according to claim 16 wherein process of analyzing the effectiveness of treatments including modalities on the results and outcomes of treating said conditions, findings, symptoms, signs, events and circumstances associate with, prior and leading to the onset of Bell's palsy comprises the process of analyzing the results and outcomes arising from patients treated with combination of treatments or modalities and without said treatments or modalities.
